(19) United States
(12) Patent Application Publication
(10) Pub. No.: US 2008/0082011 A1
(43) Pub. Date: Apr. 3, 2008

(54) Computer Input Apparatus with Health Care Function and Health Care Method

(76) Inventor: Yen-Jen Chen, Taipei City (TW)

Correspondence Address: LAW OFFICES OF LAI AND ASSOCIATES, P.C., 5800 RANCHESTER STE 200, HOUSTON, TX, 77036, US

(21) Appl. No.: 11/532,062

(22) Filed: Sep. 14, 2006

(51) Int. Cl. A61B 5/02

(52) U.S. Cl. 600/502

(57) ABSTRACT

A computer input apparatus with health care function and a health care method are disclosed. The computer input apparatus is connected to a computer through a transmission path. The computer input apparatus comprises at least a light emitting element, an optical track detection module, a measurement module and a transmission control module. A light beam is provided by the light emitting element. The optical track detection module includes a first receiving element for receiving the light beam to detect a displacement signal of the computer input apparatus. The measurement module includes a second receiving element for receiving the light beam to detect a physiology signal of fingertips.

### FIELD OF THE INVENTION

The present invention relates to a computer input apparatus with health care function and health care method, and more particularly to use a light beam emitted from a light emitting element to perform a measurement action for a fingertip portion in order to monitor a physiology situation.

### BACKGROUND OF THE INVENTION

Currently, apparatuses for use in performing a noninvasive physiology examination by using an infrared ray is quite convenience. Because the heart is the center of the circulatory system in human bodies, blood is delivered to all organs and vessels through systole and diastole. Blood and pulse statuses of a human fingertip have a close relationship with the heart functionality and the physiology situation. It is a fundamental strategy to avoid arteriosclerosis by actively handling artery health. An important parameter in diagnosing arteriosclerosis is pulse wave velocity (PWV). PWV is a ratio between distances and time differences. PWV directly relates to arteriosclerosis degrees in the measurement area. Generally speaking, when arteries are harder, the elasticity is worse and the PWV is therefore faster. Fingertip pulse waves and blood oxygen concentration can be measured by using an infrared ray measurement apparatus to emit and receive infrared rays, thereby determining the heart functionality and the physiology situation. The infrared ray measurement apparatus is usually easy to use and carry.

Referring to FIG. 1, a relationship diagram illustrates an electrocardiogram (ECG) waveform and a standard fingertip pulse wave. A longitudinal axis shows the amplitude and a horizontal axis shows the time. The relationship diagram includes the ECG waveform 110 and a fingertip pulse waveform 111 for obtaining a relationship between a heart motion and a fingertip pulse wave. The ECG waveform 110 and the fingertip pulse waveform 111 have a time difference (Q point to a starting point). T point to U point usually represents diastole. From the starting point, the waveform represents the starting systole, so pressures in vessels are increased and the amplitude also rises to a wave peak. The wave peak is the maximum amplitude of the fingertip pulse waveform 111, so the rise speed between the starting point and the wave peak can correspond to heart ejections. Furthermore, a waveform which is downward from the wave peak is called a tidal wave in the fingertip pulse waveform 111. The tidal wave reflects phenomenon, including a heart which stops ejecting, arteriectasis and the blood pressure which decreases. Subsequently, a trace wave between the tidal wave with downward direction and a dicrotic notch with upward direction is a dicrotic notch that reflects the time for artery pressures being discarded. The time is usually a boundary between systole and diastole. Lastly, the tidal wave with downward direction of the fingertip pulse waveform 111 has an obvious protruding portion that is the diastole in early-stage. Blood returns to valvular aortas to allow aorta pressures in a short run to be expanded so as to generate the waveform of the obvious protruding portion. Therefore, the fingertip pulse wave is precisely measured to determine the heart functionality and the physiology situation.

Referring to FIG. 2, a schematic diagram illustrates a conventional finger portion measurement analysis system. The system comprises a measurement body 11 and a computer 12. The measurement body 11 in a predetermined time performs a measurement action for a fingertip portion, thereby generating a measurement result. The computer 12 is electrically connected to the measurement body 11 for receiving the measurement result and processing the measurement result in order to obtain health information such as pulses, oxygen content in blood and blood pressures. The measurement body must be electrically connected to the computer while measuring. The measurement body is removed from the computer while is not in use. The aforesaid way causes inconvenience.

Referring to FIG. 3, a schematic diagram illustrates a conventional finger cot health detector. The finger cot health detector comprises a finger cot measurement body 21 and a surveillance main body 22. The finger cot measurement body 21 performs a measurement action for a fingertip portion, thereby generating a measurement result. The surveillance main body 22 is electrically connected to the finger cot measurement body 21 for obtaining health information such as pulses, blood oxygen concentration and blood pressures based on the health information. The surveillance main body 22 can be fastened to a wrist so as to easily carry and use. For example, the finger cot measurement body 21 can detect the variation of wavelength of reflected infrared ray due to different blood oxygen concentrations when blood is irradiated by an infrared ray. The reflected infrared ray is further analyzed by the surveillance main body 22 to obtain blood oxygen concentrations. However, the health information obtained by the surveillance main body 22 may not be stored in a large number. Health information tracing function may not be provided.

To overcome the foregoing shortcomings, the inventor of the present invention based on years of experience to conduct extensive researches and experiments invents a computer input apparatus with health care function and health care method. The conventional concept of using an infrared ray to measure a fingertip portion is extended. The computer input apparatus as an optical mouse is taken to be a measurement device. A transmission path between a computer and the computer input apparatus is used to transmit a physiology signal of the fingertip portion to the computer, thereby obtaining related health information. Therefore, connecting and removing the measurement body is unnecessary. The computer is also used to store the health information to be conveniently traced and recorded. The computer may not be influenced while in use. Furthermore, the present invention is also used to achieve the goal of health care even though an operation end is working.

### SUMMARY OF THE INVENTION

A primary object of the present invention is to provide a computer input apparatus with health care function and health care method, wherein the computer input apparatus is connected to a computer through a transmission path. The computer input apparatus is taken to be an apparatus for measuring. The transmission path between the computer and the computer input apparatus is also used to transmit a physiology signal of a fingertip portion to the computer. The physiology signal of the fingertip portion is then analyzed to obtain health information to achieve the health care effect.

The computer input apparatus with health care function comprises a light emitting element, an optical track detection module, a measurement module and a transmission control module. The light emitting element is for emitting a light beam. The optical track detection module has a first receiving element for receiving an infrared ray and a red light to detect a displacement signal of the computer input apparatus. The measurement module has a second receiving element for receiving the infrared ray and the red light to measure the physiology signal of the fingertip portion. The transmission control module transmits the physiology signal of the fingertip portion or the displacement signal of the computer input apparatus to the computer through the transmission path between the computer and the computer input apparatus. In addition, an analysis unit is disposed in the computer. Alternatively, the analysis unit is electrically connected to the computer by using a universal serial bus (USB) to analyze the physiology signal of the fingertip portion so as to generate health information like oxygen content in blood, blood pressures and pulses. A health index is further generated based on the health information. Moreover, the computer input apparatus can use the computer to record the health information and the health index that are provided to the analysis unit to perform the tracing and statistics. The display unit of the computer displays the health information and the health index. A remote medical treatment is further achieved by connecting internet and family physicians or hospitals.

Other features and advantages of the present invention and variations thereof will become apparent from the following description, drawings, and claims.

### BRIEF DESCRIPTION OF THE DRAWINGS

FIG. 1 is a relationship diagram illustrating an electrocardiogram waveform and a standard fingertip pulse wave;

FIG. 2 is a schematic diagram illustrating a conventional fingertip portion measurement analysis system;

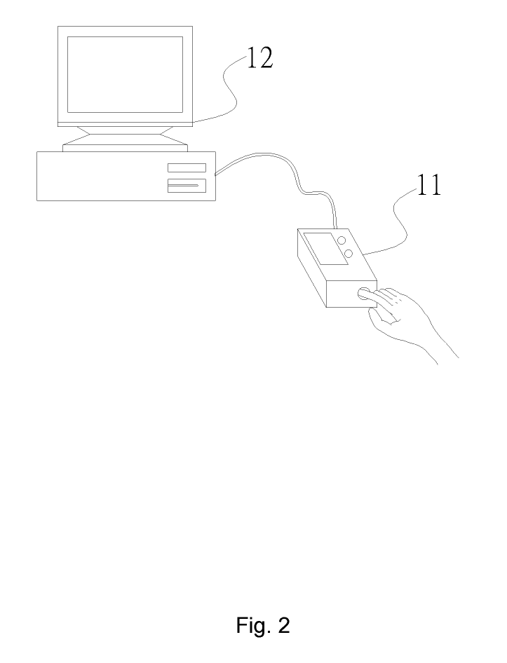

FIG. 3 is a schematic diagram illustrating a conventional finger cot health detector;

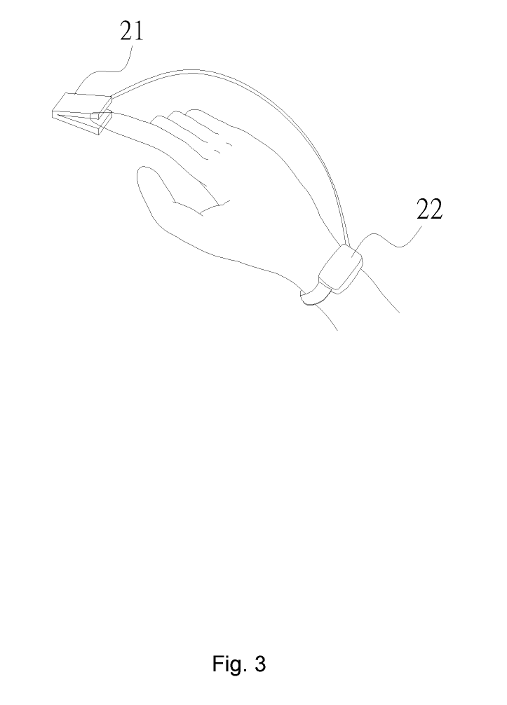

FIG. 4 is a block diagram illustrating a computer input apparatus with health care function of the present invention;

FIG. 5 is a schematic diagram illustrating a computer input apparatus with health care function of the present invention;

FIG. 6 is an oscillogram illustrating a measurement module for use in measuring a fingertip of the present invention; and

FIG. 7 is a flowchart illustrating a health care method of the present invention.

### DETAILED DESCRIPTION OF THE PREFERRED EMBODIMENTS

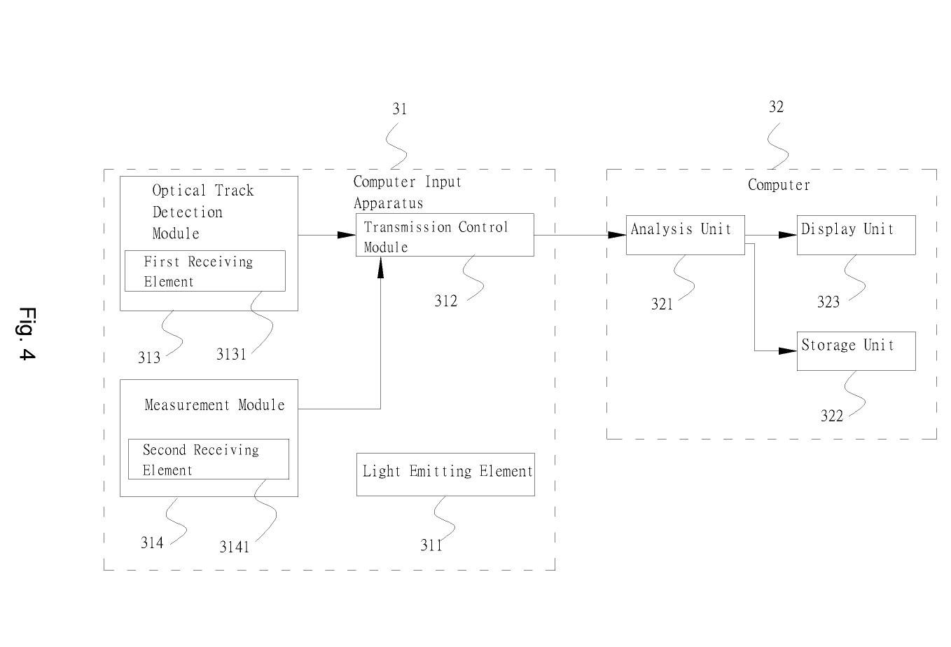

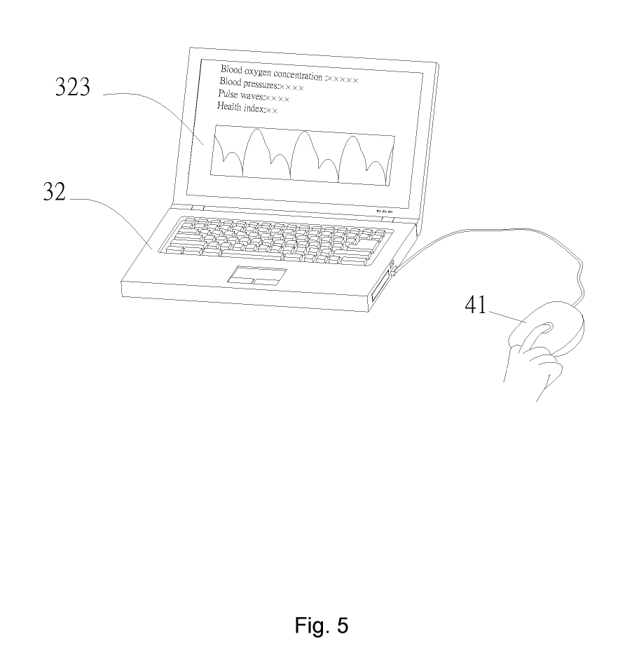

Referring to FIG. 4 and FIG. 5, FIG. 4 is a block diagram illustrating a computer input apparatus with health care function of the present invention. FIG. 5 is a schematic diagram illustrating a computer input apparatus with health care function of the present invention. As shown in FIG. 4, the computer input apparatus 31 comprises a light emitting element 311, an optical track detection module 313, a measurement module 314 and a transmission control module 312. A computer 32 comprises an analysis unit 321, a storage unit 322 and a display unit 323.

The light emitting element 311 is for emitting a light beam as an infrared ray and a red light. The optical track detection module 313 has a first receiving element 3131 for receiving the infrared ray or the red light, thereby detecting a displacement signal of the computer input apparatus. The measurement module 314 has a second receiving unit 3141 for receiving the infrared ray and the red light, which are reflected by fingers. The measurement module can filter noise of reflected light beam. A signal which has been filtered is then amplified. Because the infrared ray and the red light can be absorbed by unoxygenated blood and oxygenated blood separately, reflected wavelengths and amplitudes are then changed based on different physiology situations. The measurement module 314 then uses a noninvasive way to measure a physiology signal of a fingertip portion. As shown in FIG. 5, when the computer input apparatus 31 is an optical mouse 41, the light beam of the displacement signal of the optical mouse 41 is detected to measure the physiology situation of the fingertip portion. The transmission control module 312 transmits the displacement signal of the computer input apparatus or the physiology signal of the fingertip portion to the computer 32 by using a transmission path between the computer input apparatus 31 and the computer 32. The analysis unit 321 then analyzes the physiology signal of the fingertip portion. An arithmetic processing is performed for the physiology signal of the fingertip portion based on a predetermined standard to generate health information like oxygen content in blood, blood pressures and pulses. A health index is further generated based on the health information. The analysis unit 321 is a processing program in the computer 32. The storage unit 322 provides a recording function to record the health information and the health index. The analysis unit 321 then performs the tracing and statistics. The display unit 323 displays the health information and the health index to allow an operation end to perform a personal health care action. The display unit 323 is preferably a display screen. In addition, the computer 32 can be connected to internet for transmitting the health information and the health index to family physicians or hospitals, thereby accomplishing a remote medical treatment function.

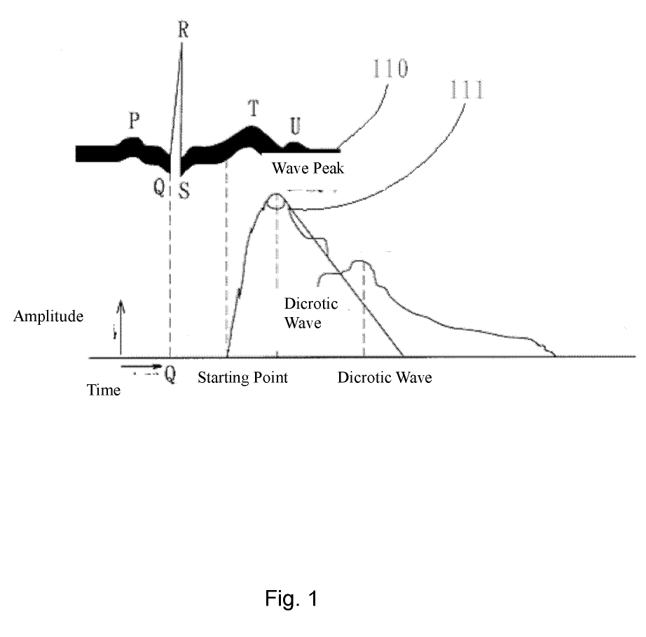

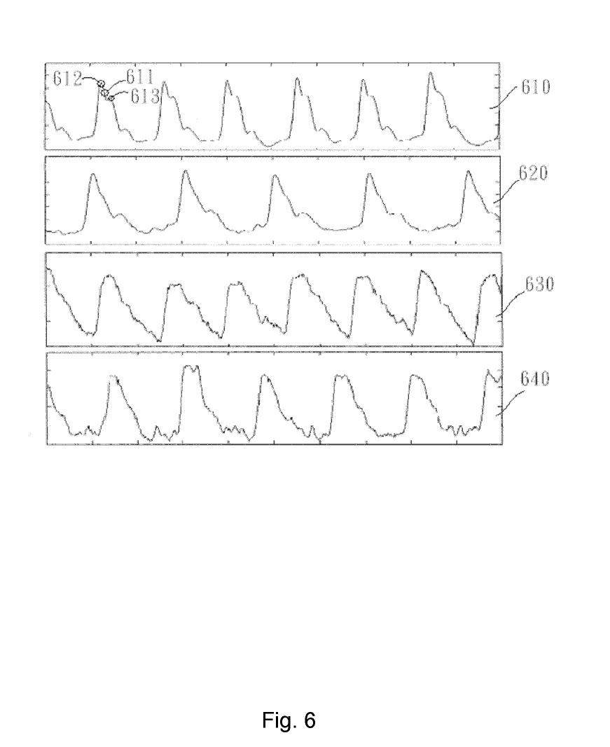

Referring to FIG. 6, an oscillogram illustrates fingertips measured by a measurement module of the present invention. An oscillogram 610 and an oscillorgam 620 are obtained separately by measuring a fingertip of a young man and a fingertip of a middle-aged man. Vascular elasticity of the middle-aged man may be worse by corresponding to tidal waves 611 of the oscillogram 610 and the oscillogram 620. Moreover, an oscillogram 630 is obtained by measuring the fingertip of the middle-aged man suffered from hypertension. The oscillogram 640 is obtained by measuring the fingertip of an elder suffered from hypertension, hyperlipidemias and nephropathy The oscillograms 630 and 640 are compared with the standard fingertip pulse waveform 111 as shown in FIG. 1 so as to know the oscillorgam produced by measuring a fingertip of a person suffered from cardiovascular diseases that a wave peak 612 is therefore wider, and the tidal wave 611 and a dicrotic wave 613 are unclear. Hence, the analysis unit 321 can use these waves to analyze and perform arithmetic processing.

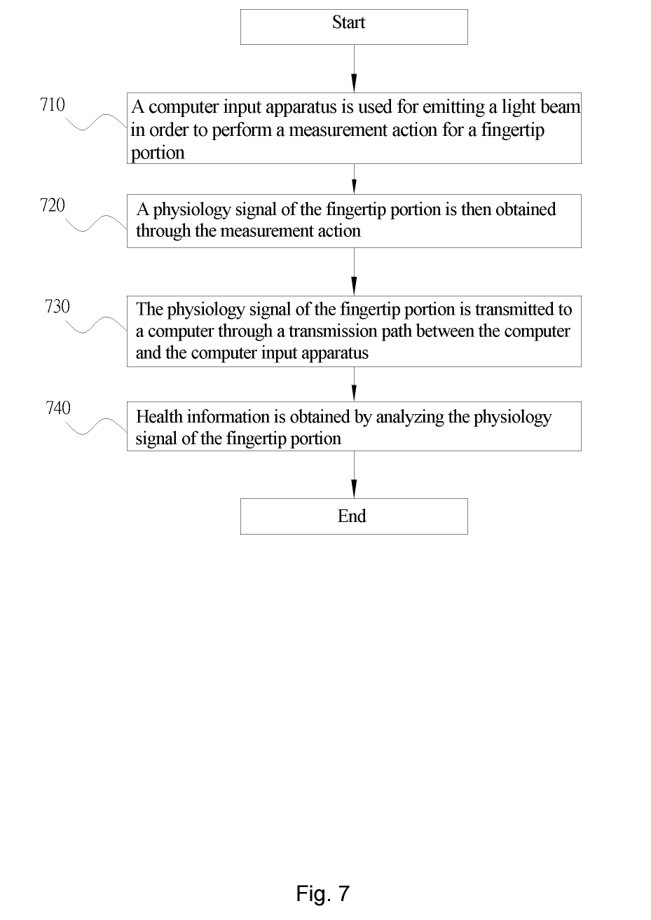

In accordance with the computer input apparatus with health care, FIG. 7 is a flowchart illustrating a health care method. The method comprises the following steps:

Step 710: A computer input apparatus is used for emitting a light beam in order to perform a measurement action for a fingertip portion.

Step 720: A physiology signal of the fingertip portion is then obtained through the measurement action.

Step 730: The physiology signal of the fingertip portion is transmitted to a computer through a transmission path between the computer and the computer input apparatus.

Step 740: Health information is obtained by analyzing the physiology signal of the fingertip portion.

In those steps, an optical mouse is taken to be the computer input apparatus. The measurement action is then performed and the displacement signal of the optical mouse is then detected through the light beam (e.g. an infrared ray and a red light) emitted by the optical mouse. Moreover, by using the transmission path between the computer and the computer input apparatus, the physiology signal of the fingertip portion is transmitted to the computer. The computer then knows the displacement signal of the optical mouse. In addition, a health index is further obtained to provide a user for references by analyzing the health index.

Although the features and advantages of the embodiments according to the preferred invention are disclosed, it is not limited to the embodiments described above, but encompasses any and all modifications and changes within the spirit and scope of the following claims.

What is claimed is:

1. A computer input apparatus with health care function connected to a computer through a transmission path, and said computer input apparatus comprising:
a light emitting element for providing a light beam;
an optical track detection module having a first receiving element for receiving said light beam to detect a displacement signal of said computer input apparatus;
a measurement module having a second receiving element for receiving said light beam to measure a physiology signal of a fingertip portion; and
a transmission control module for transmitting said physiology signal of said fingertip portion or said displacement signal of said computer input apparatus to said computer through said transmission path.

2. The computer input apparatus with health care of claim 1, wherein said computer input apparatus is an optical mouse.

3. The computer input apparatus with health care function of claim 1, wherein said light beam is an infrared ray.

4. The computer input apparatus with health care function of claim 1, wherein said light beam is a red light.

5. The computer input apparatus with health care function of claim 1, wherein said physiology signal of said fingertip portion is transmitted to an analysis unit in said computer, and said analysis unit is for analyzing said physiology signal of said fingertip portion to generate health information.

6. The computer input apparatus with health care function of claim 5, wherein said analysis unit is disposed in said computer.

7. The computer input apparatus with health care function of claim 5, wherein said analysis unit is electrically connected to said computer by using a universal serial bus (USB).

8. The computer input apparatus with health care function of claim 5, wherein said analysis unit further uses said health information to provide at least one health index.

9. The computer input apparatus with health care function 5, wherein said health information includes oxygen content in blood, blood pressures and pulses.

10. The computer input apparatus with health care function of claim 1, wherein said computer includes a display unit for displaying said health information.

11. The computer input apparatus with health care of claim 5, wherein said health information is recorded in a storage unit.

12. The computer input apparatus with health care of claim 11, wherein said storage unit is disposed in said computer in order to record said health information.

13. The computer input apparatus with health care of claim 11, wherein said storage unit is electrically connected to said computer by using a USB to record said health information.

14. The computer input apparatus with health care of claim 5, wherein said computer uses internet to transmit said health information.

15. A health care method for providing a health care function by using a computer and a computer input apparatus, and said health care method comprising:
using said computer input apparatus to provide a light beam so as to perform a measurement action for a fingertip portion;
obtaining at least one physiology signal of said fingertip portion through said measurement action;
transmitting said physiology signal of said fingertip portion to said computer through a transmission path between said computer and said computer input apparatus; and
analyzing said physiology signal of said fingertip portion to obtain health information.

16. The health care method of claim 15, further comprising the step of using an optical mouse to be said computer input apparatus.

17. The health care method of claim 16, further comprising the step of using said light beam to detect a displacement signal of said optical mouse.

18. The health care method of claim 15, further comprising the step of using said transmission path to allow said computer to know said displacement signal.

19. The health care method of claim 15, further comprising the step of analyzing said health information to provide at least one health index.

20. The health care method of claim 15, further comprising the step of using an infrared ray to be said light beam.

21. The health care method of claim 15, further comprising the step of using a red light to be said light beam.
